(19) United States
(12) Patent Application Publication
(10) Pub. No.: US 2009/0053154 A1
(43) Pub. Date: Feb. 26, 2009

(54) MIXTURES OF ETHYLHEXYL P-METHOXYCINNAMATE AND ETHYLHEXYL SALICYLATE

(75) Inventor: Roland Langner, Bevern (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/089,543

(22) PCT Filed: Aug. 28, 2006
(86) PCT No.: PCT/EP2006/065705

§ 371 Date: Aug. 29, 2008

Related U.S. Application Data
(60) Provisional application No. 60/726,243, filed on Oct. 14, 2005.

(51) Int. Cl. A61K 8/368; A61K 31/60; A61Q 17/04

(52) U.S. Cl. 424/60; 514/163

(57) ABSTRACT

The present invention relates to a composition comprising or (preferably) consisting of 2-ethylhexyl p-methoxycinnamate and 2-ethylhexyl salicylate, the sum of the constituents (a) and (b) ranging from 50 to 100 wt. %, based on the total weight of the composition.

The present invention relates to a (synergistically active) composition comprising or consisting of the two UV filters ethylhexyl p-methoxycinnamate and ethylhexyl salicylate, to the use of this composition in cosmetic or dermatological formulations for protecting the skin and hair from damaging UV rays, especially light protection formulations and agents, and to a process for the preparation of corresponding cosmetic or dermatological formulations.

UV filters (UV absorbers) are compounds with a pronounced absorption capacity for ultraviolet radiation. They are used particularly in cosmetic and dermatological formulations for protecting the skin and hair. UV filters are also used to improve the light stability of products.

UV rays are divided according to wavelength into UV-A rays (320-400 nm), UV-B rays (280-320 nm) and UV-C rays (<280 nm).

UV rays can cause acute and chronic skin damage, the type of damage depending on the wavelength of the radiation. Thus UV-B rays can cause sunburn (erythema) or even very severe skin burns. UV-A rays penetrate deeper skin layers, where they can accelerate the skin's ageing process (known as photo ageing). UV rays can also trigger phototoxic or photoallergic skin reactions. UV-C rays, on the other hand, do not reach the earth's surface.

Very frequent and unprotected irradiation of the skin with sunlight leads to a loss of elasticity, increased wrinkling and hence premature ageing of the skin. Pathological skin changes or even skin cancer can be caused in extreme cases.

UV filters are divided into UV-A and UV-B filters according to the position of their absorption maxima. A UV broadband absorber is a UV filter that can absorb UV-B and UV-A rays simultaneously.

2-Ethylhexyl p-methoxycinnamate (ethylhexyl p-methoxycinnamate, CAS no.: 5466-77-3; USAN: Octinoxate, Neo Heliopan® AV (Symrise trade mark); hereafter abbreviated to EHMC) is a liquid, oil-soluble UV-B filter with a specific extinction E 1%/1 cm at 308 nm of approx. 850 (cf. H&R Contact no. 51, January 1991, pages 3-8). 2-Ethylhexyl p-methoxycinnamate is also frequently abbreviated to octyl methoxycinnamate (OMC) in the literature.

2-Ethylhexyl salicylate (CAS no.: 118-60-5; USAN: Octisalate, Neo Heliopan® OS (Symrise trade mark); hereafter abbreviated to EHS) is likewise a liquid, oil-soluble UV-B filter with a specific extinction E 1%/1 cm at 305 nm of approx. 180 (cf. H&R Contact no. 51, January 1991, pages 3-8). The specific extinction of EHS is lower than that of EHMC by a factor of about 5.

The specific extinction of UV filters is of primary importance as an index for assessing the efficacy in respect of the achievable light protection factor (LPF, hereafter also SPF (sun protection factor)). The higher the specific extinction, the more effective is the protection of the skin from damaging UV rays. The light protection factor of a (light protection) formulation is a measure of the UV-B protection it can achieve. The absolute SPF values also depend within certain limits on the cosmetic base formulation used.

Measurement of the light protection factor is now standardized and normalized by the so-called COLIPA method, which is conventional and valid throughout Europe, and the irradiation spectrum and output of the solar simulator used to determine the SPF are accurately defined. The amount applied and the type of product application are also prescribed. This test method is independent of skin type and the age of the test subjects. The COLIPA method is a test procedure that yields reproducible results.

Cosmetic or dermatological formulations quite frequently contain a combination of several different UV filters. Use of the two UV-B filters EHMC and EHS together in light protection formulations is also widespread, especially in the USA. These two UV-B filters are normally combined with other, different UV-B and UV-A filters.

Unless indicated otherwise, the percentages below are by weight.

A light protection formulation comprising 3 wt. % of EHMC and 5 wt. % of EHS is described e.g. in “Sunscreens—Development, Evaluation and Regulatory Aspects”, edited by N. Lowe, N. Shaath, Marcel Dekker, Inc. New York, chapter 16, page 263.

“Chemistry and Technology of the Cosmetics and Toiletries Industry”, edited by D. F. Williams, W. H. Schmitt, Blackie Academic & Professional, London, chapter 3.8, page 132, refers to a light protection formulation which, in addition to 7.5 wt. % of EHMC and 5 wt. % of EHS, contains another organic UV filter (6 wt. % of oxybenzene) and 10 wt. % of the SPF booster octyldodecyl neopentanoate.

According to Parfümerie und Kosmetik, April 1999, 36-39, an in vitro measurement of an emulsion containing 5 wt. % of EHMC gave an SPF of 10.1. This article takes account of the boosting action on the light protection factor and an increase in the UV-A/UV-B ratio and in the critical wavelength of emulsions containing organic UV filters due to zinc oxide. Explicit reference is made to a synergistic effect of mixtures of zinc oxide and EHMC on the light protection factor. A cosmetic formulation containing 5 wt. % of EHMC, 3 wt. % of EHS, 2 wt. % of benzophenone and 4.6 wt. % of zinc oxide is mentioned in which the zinc oxide substantially increases the UV-A/UV-B ratio and the critical wavelength.

The SPF-boosting actions of titanium dioxide, dicaprylyl maleate and microfine polytetrafluoroethylene are also described in the literature.

Developers of cosmetic or dermatological formulations, especially light protection formulations, are constantly endeavouring to prepare very efficient recipes with suitable combinations of UV filters. All the more surprisingly and unpredictably for those skilled in the art, it has now been found that the UV-B filters EHMC and EHS give rise to a synergistic action on the resulting light protection factor (total light protection factor).

It was surprising and unpredictable that a composition containing EHMC and EHS has a significantly higher (synergistically boosted) light protection factor than the arithmetic mean of the separately determined light protection factors of the UV filters EHMC and EHS at the same dosage in each case. Because of the markedly higher UV absorption and markedly higher light protection factor of EHMC compared with EHS, a lower light protection factor would have been expected for a composition according to the invention. However, it has been found that the light protection factor is even higher than with EHMC alone.

The light protection factors were determined in vivo by the COLIPA method conventionally used in industry (status: February 2003). The compositions according to the invention were used in the in vivo tests in the form of an O/W emulsion based on a cosmetically acceptable emulsion base.

It is taught according to the invention to use 2-ethylhexyl p-methoxycinnamate to synergistically boost the light protection factor of a composition containing 2-ethylhexyl salicylate. It is further taught according to the invention to use 2-ethylhexyl salicylate to synergistically boost the light protection factor of a composition containing 2-ethylhexyl p-methoxycinnamate.

The present invention therefore provides compositions comprising or consisting of EHMC and EHS which, in cosmetic or dermatological (light protection) formulations, have a synergistic action on the resulting light protection factors.

The invention therefore provides a composition comprising or, preferably, consisting of

(a) 2-ethylhexyl p-methoxycinnamate and

(b) 2-ethylhexyl salicylate,

the sum of the constituents (a) and (b) ranging from 50 to 100 wt. %, based on the total weight of the composition.

The compositions according to the invention make it possible advantageously to utilize the synergism described above. In particular, such compositions facilitate handling in the preparation of cosmetic or dermatological (light protection) formulations, thereby saving time and costs.

The invention also provides the use of (synergistically active) compositions according to the invention for use in cosmetic or dermatological (light protection) formulations.

Preferably, each individual constituent (a) and (b) is present in an amount of at least 3 wt. %, based on the total weight of the composition.

The sum of the constituents (a) and (b) ranges preferably from 90 to 100 wt. % and particularly preferably from 95 to 100 wt. %, based on the total weight of the composition.

In one preferred embodiment, a composition according to the invention does not contain any other UV filters apart from the constituents EHMC and EHS, i.e. neither organic UV-A or UV-B filters, nor organic broadband filters, nor pigmentary UV filters, e.g. zinc oxide or titanium dioxide.

In another preferred embodiment, a composition according to the invention does not contain any other SPF-boosting substances, especially zinc oxide, titanium dioxide, octyldodecyl neopentanoate, dicaprylyl maleate or microfine polytetrafluoroethylene.

Any weight ratio of EHMC and EHS is advantageous in a composition according to the invention, the weight ratio of EHS to EHMC preferably ranging from 10:1 to 1:10. The weight ratio of EHS to EHMC ranges preferably from 4:1 to 1:4 and particularly preferably from 2:1 to 1:2, the SPF-increasing action being the most pronounced at these weight ratios.

To achieve an even greater protection of the human epidermis and also human hair from damaging UV rays, ready-to-use cosmetic or dermatological (light protection) formulations can contain other, different conventional UV filters in addition to a composition according to the invention.

Cosmetic or dermatological (light protection) formulations are normally prepared by first preparing several partial formulations (e.g. an oil phase and a water phase) separately and then mixing them together, the respective cosmetic ingredients being incorporated separately and successively into the appropriate partial formulations.

The compositions of EHMC and EHS according to the invention in a ready-to-use cosmetic or dermatological (light protection) formulation are preferably proportioned so that the amount of EHMC ranges from 0.1 to 20 wt. %, preferably from 0.5 to 10 wt. %,

and/or
the amount of EHS ranges from 0.1 to 10 wt. %, preferably from 0.5 to 6 wt. %, based in each case on the total weight of the ready-to-use cosmetic or dermatological (light protection) formulation.

Examples of other suitable conventional cosmetic UV filters are organic UV filters from the class comprising 4-aminobenzoic acid and derivatives; salicylic acid derivatives; benzophenone derivatives; dibenzoylmethane derivatives, diphenyl acrylates, 3-imidazol-4-ylacrylic acid and its esters, benzofuran derivatives; benzylidenemalonate derivatives; cinnamic acid derivatives; camphor derivatives; trianilino-s-triazine derivatives; 2-hydroxyphenylbenzotriazole derivatives; 2-phenylbenzimidazole-5-sulfonic acid and its salts; menthyl anthranilate; benzotriazole derivatives; polymeric UV filters containing one or more organosilicon radicals; and finely divided zinc oxide and titanium dioxide. A review of the various UV filters for use in cosmetic or dermatological (light protection) formulations can be found inter alia in the following publications: CTFA Dictionary; Council Directive 76/768/EC, Annex 7, Part 1; Federal Register May 21, 1999 (64 FR 27666)/USA.

There follows an Application Example of the use of the composition of EHMC and EHS according to the invention in a cosmetic light protection formulation. The raw materials and active substances in the Formulation Example are given with the respective INCI (International Nomenclature of Cosmetic Ingredients) name.

The Example which follows illustrates the invention.

### Light Protection Formulations: Sunscreen Emulsions (O/W Emulsions)

Four ready-to-use light protection formulations L1-L4, each containing 10 wt. % of the UV filters EHMC and/or EHS, were prepared.

| Part | Raw material | INCI name | wt. % |
|---|---|---|---|
| A | Dracorin GOC | Glyceryl oleate citrate, caprylic/capric | 2.00 |
| | | triglyceride | |
| | Dracorin GMS | Glyceryl stearate | 0.50 |
| | PCL solid | Stearyl heptanoate, stearyl caprylate | 0.50 |
| | EHMC and/or EHS | Ethylhexyl methoxycinnamate and/or | 10.00 |
| | | ethylhexyl salicylate | |
| | Lanette O | Cetearyl alcohol | 1.00 |
| | Tegosoft TN | C12-15 alkyl benzoate | 3.00 |
| | Dow Corning 246 fluid | Cyclohexasiloxane, cyclopentasiloxane | 1.00 |
| | EDTA BD | Disodium EDTA | 0.10 |
| | Copherol 1250 | Tocopherol acetate | 0.50 |
| | Carbopol Ultrez 21 | Acrylates/C10-30 alkyl acrylate | 0.25 |
| | | crosspolymer | |
| | Keltrol RD | Xanthan gum | 0.15 |
| B | Demineralized water | Water (aqua) | 74.50 |
| | Dragocid liquid | Phenoxyethanol (and) methylparaben | 0.80 |
| | | (and) ethylparaben (and) butylparaben | |
| | | (and) propylparaben (and) | |
| | | isobutylparaben | |
| | Glycerin 99% | Glycerin | 4.70 |
| | Potassium sorbate | Potassium sorbate | 0.10 |
| C | Sodium hydroxide | Sodium hydroxide | 0.70 |
| | solution, 10% aq. | | |
| | Perfume oil | Fragrance (perfume) | 0.20 |

### Preparatory Instructions:

Part A: Heat all the raw materials, except Keltol RD and Carbopol Ultrez 21, to 75° C. to 80° C. and dissolve; then add Keltrol and Carbopol and subsequently homogenize briefly.

Part B: Dissolve raw materials in water, stir the solution into part A and then homogenize briefly.

Part C: At approx. 35° C. to 40° C., stir the 10% sodium hydroxide solution and the perfume oil successively into the emulsion of part A and part B and then homogenize briefly.

Finally, stir to room temperature and fill into suitable packs.

The light protection factors of the sunscreen emulsions obtained in this way were measured on 12 test subjects by the COLIPA method of SPF determination (status: February 2003).

The following Table shows the amounts of EHMC and/or EHS contained in the four light protection formulations L1-L4 and the corresponding SPF determined in vivo.

| | L1 | L2 | L3 | L4 |
|---|---|---|---|---|
| EHMC | 10 wt. % | — | 5 wt. % | 6.7 wt. % |
| EHS | — | 10 wt. % | 5 wt. % | 3.3 wt. % |
| In vivo SPF | 11.2 | 7.1 | 11.7 | 11.6 |

1-11. (canceled)

12. A composition comprising
(a) 2-ethylhexyl p-methoxycinnamate and
(b) 2-ethylhexyl salicylate,
wherein the sum of the constituents (a) and (b) ranges from 50 to 100 wt. %, based on the total weight of the composition.

13. The composition according to claim 12 wherein each individual constituent (a) and (b) is present in an amount of at least 3 wt. %, based on the total weight of the composition.

14. The composition according to claim 12, wherein the sum of the constituents (a) and (b) ranges from 90 to 100 wt. % based on the total weight of the composition.

15. The composition according to claim 12, wherein the sum of the constituents (a) and (b) ranges from 95 to 100 wt. % based on the total weight of the composition.

16. The composition according to claim 15, wherein the composition consists essentially of the constituents (a) and (b).

17. The composition according to claim 12, wherein the weight ratio of (a) to (b) ranges from 10:1 to 1:10.

18. The composition according to claim 12, wherein the composition has a synergistically boosted light protection factor.

19. The composition according to claim 12, characterized in that it does not comprise any other SPF-boosting substances.

20. A process for the preparation of a cosmetic or dermatological formulation comprising the following steps:
preparation of a composition according to claim 12 and
bringing this composition into contact with, or mixing this composition with, the remaining constituents of the cosmetic or dermatological formulation.

21. A method for preventing skin damage in a subject comprising applying the composition according to claim 12 to the skin of said subject as a UV filter.

22. A method for protecting the skin and hair in a subject comprising topically applying the composition according to claim 12 in a cosmetic or dermatological formulation.

23. A method for synergistically boosting the light protection factor of a composition containing 2-ethylhexyl salicylate comprising adding 2-ethylhexyl p-methoxycinnamate.

24. A method for synergistically boosting the light protection factor of a composition containing 2-ethylhexyl p-methoxycinnamate comprising adding 2-ethylhexyl salicylate.
